(19) United States
(12) Patent Application Publication
(10) Pub. No.: US 2005/0090824 A1
(43) Pub. Date: Apr. 28, 2005

(54) Method and surgical tool for inserting a longitudinal member

(75) Inventors: Alan E. Shluzas, West Roxbury, MA (US); Alexander J. Curry, Attleboro, MA (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: Endius Incorporated

(21) Appl. No.: 10/972,156

(22) Filed: Oct. 22, 2004

Related U.S. Application Data
(60) Provisional application No. 60/513,462, filed on Oct. 22, 2003.

(51) Int. Cl. A61F 2/30; A61B 17/60; A61F 2/38

(52) U.S. Cl. 606/61; 606/99

(57) ABSTRACT

A method for inserting a longitudinal member into a body of a patient includes holding the longitudinal member in a first position with a surgical tool. The longitudinal member is moved through an access port in the first position with the surgical tool. The longitudinal member is rotated from the first position into a second position extending transverse to the first position without releasing the longitudinal member from the surgical tool. The surgical tool includes a holding device for holding the longitudinal member in a first position. An articulating device rotates the longitudinal member from the first position into the second position without releasing the longitudinal member.

### RELATED APPLICATION

This application claims the benefit of U.S. Provisional Application No. 60/513,462, filed Oct. 22, 2003 and is incorporated herein by reference.

### FIELD OF THE INVENTION

The present invention relates to a method and a surgical tool for inserting a longitudinal member into a body of a patient, and more specifically, to a method and a surgical tool for inserting a longitudinal member during a minimally invasive surgery.

### BACKGROUND OF THE INVENTION

It is known to connect a longitudinal member to vertebrae of a spinal column during a minimally invasive surgical procedure. The longitudinal member is moved through an access port, a tubular structure, a retractor, or a cannula into the body of a patient using a surgical tool. The longitudinal member is released by the surgical tool while inside the body. The longitudinal member is then picked up for insertion into connectors connected to the vertebrae.

### SUMMARY OF THE INVENTION

The present invention relates to a method and a surgical tool for inserting a longitudinal member into a body of a patient. The longitudinal member is held in a first position with the surgical tool. The longitudinal member is moved through an access port, a tubular structure, a retractor, or a cannula in the first position with the surgical tool. The longitudinal member is rotated from the first position into a second position extending transverse to the first position without releasing the longitudinal member from the surgical tool.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of a surgical tool for inserting a longitudinal member or a spine rod into a body of a patient;

FIG. 2A is an exploded view of a portion of the surgical tool of FIG. 1;

FIG. 2B is an exploded view of another portion of the surgical tool of FIG. 1;

FIG. 3 is a sectional view of the surgical tool of FIG. 1 showing the tool holding a spine rod for insertion into the body of a patient;

FIG. 4 is a sectional view of the surgical tool of FIG. 1 showing the tool in position for releasing the spine rod;

FIG. 5 is a schematic illustration of the surgical tool of FIG. 1 inserting the spine rod into the body of a patient; and

FIG. 6 is a schematic illustration of the surgical tool of FIG. 1 inserting the rod into fasteners connected to vertebrae of a spinal column.

### DESCRIPTION OF THE INVENTION

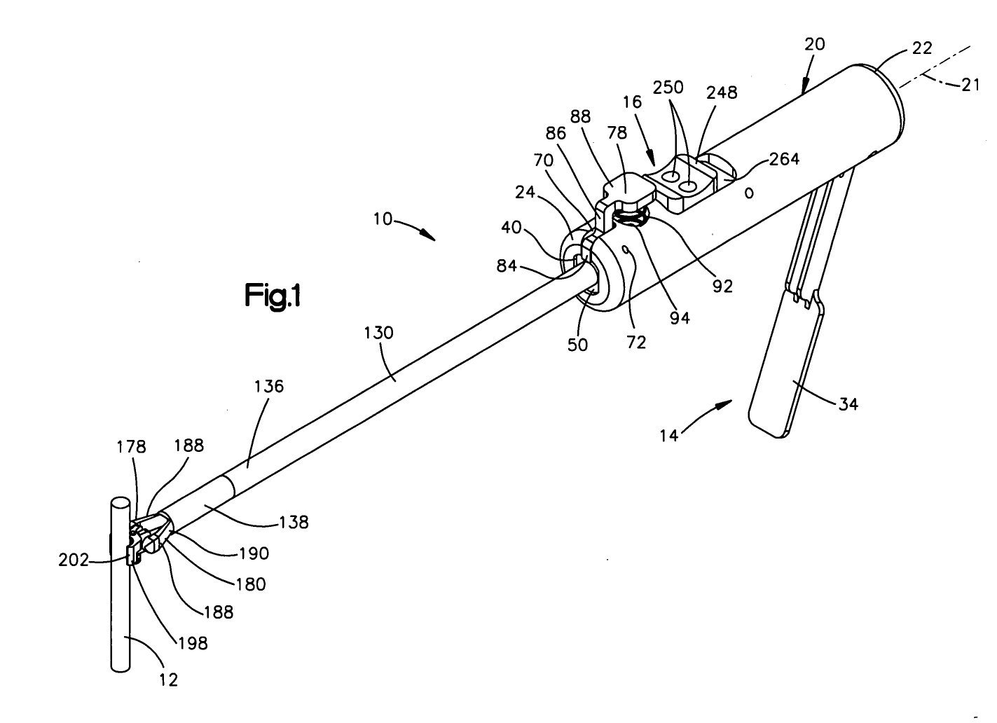

FIG. 1 is a perspective view of a surgical tool 10 for inserting a longitudinal member or spine rod 12 into the body of a patient. The tool 10 may be used to insert the rod 12 through an access port, a tubular structure, a retractor, or a cannula during a minimally invasive surgery. The spine rod 12 may be connected to vertebrae V of a spinal column to retain the vertebrae in a desired spatial relationship.

The surgical tool 10 includes a holding device 14 and an articulating device 16. The holding device 14 holds the rod 12 in a first position during insertion of the rod. The articulating device rotates the rod 12 from the first position into a second position extending transverse to the first position.

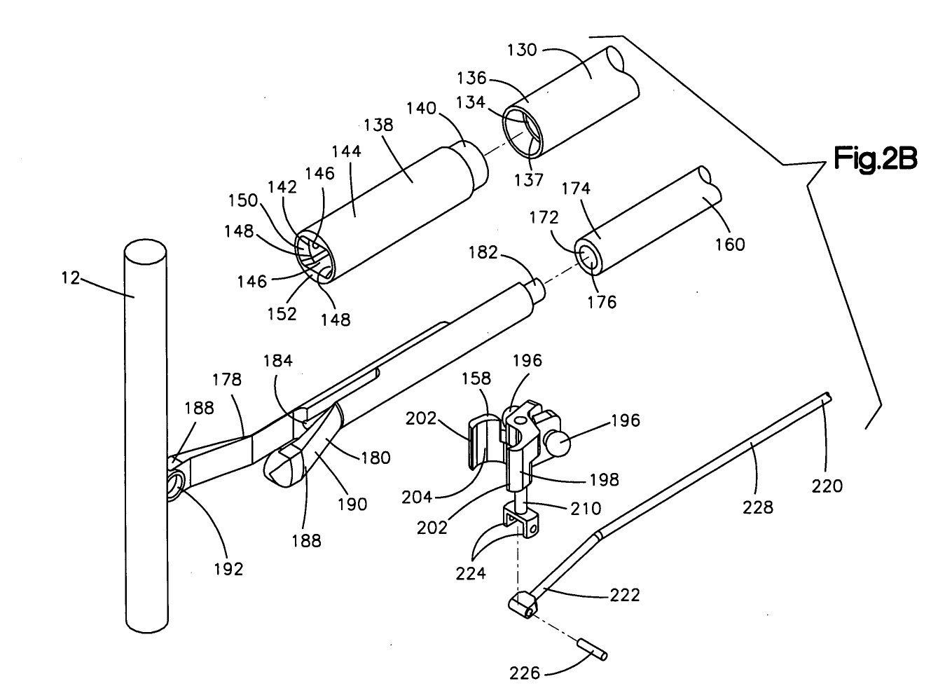

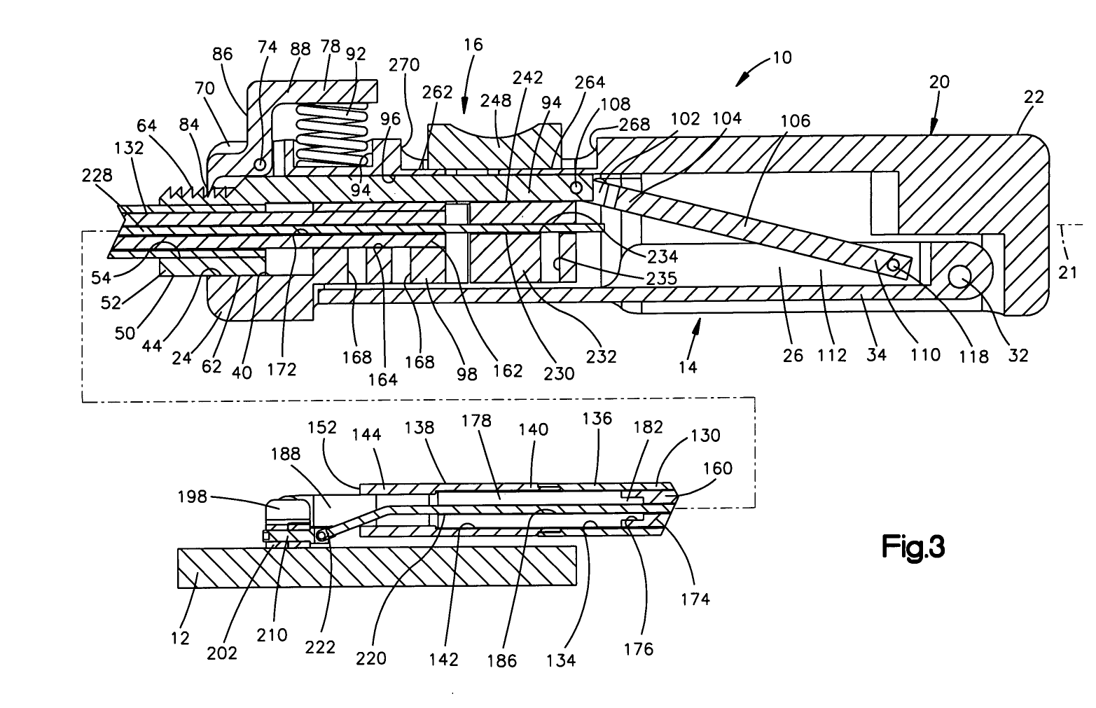

The holding device 14 includes a fixed handle 20 having a longitudinal axis 21. The fixed handle 20 has a first proximal end 22 and a second distal end 24. The handle 20 (FIGS. 3 and 4) includes a recess 26 in a lower portion of the handle 20. The recess 26 extends axially from adjacent the first end 22 toward the distal end 24.

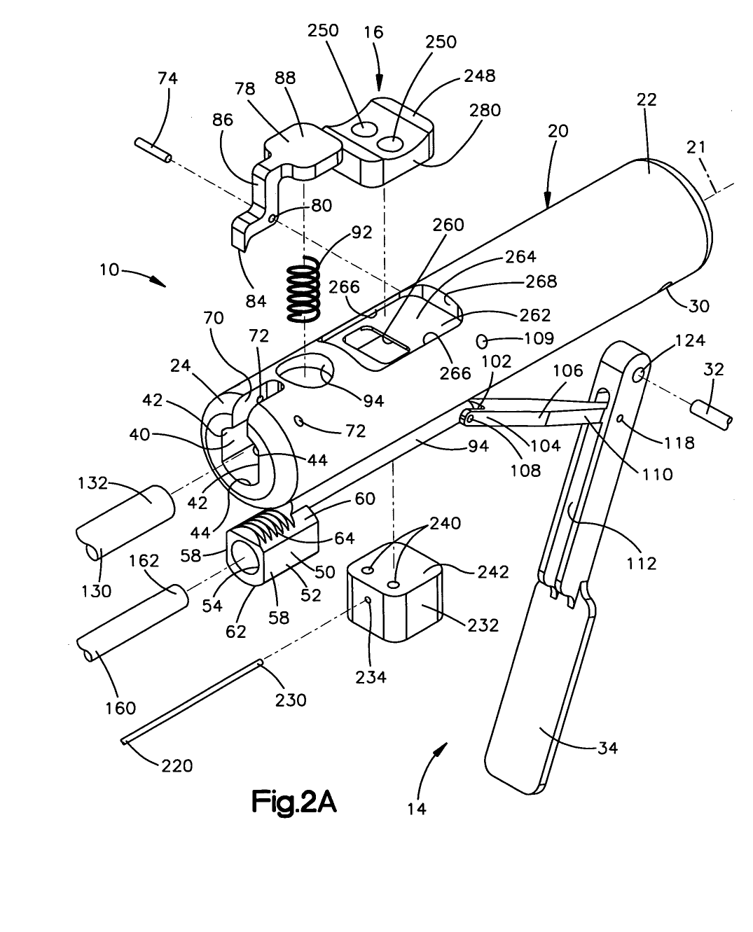

A pair of openings 30, one of which is shown in FIG. 2A, extends through the proximal end 22 and intersect the recess 26. A pin 32 extends through the openings 30 into the fixed handle 20 to pivotally connect a lever 34 to the proximal end 22 of the fixed handle 20. When the lever 34 is pivoted into the recess 26 in the fixed handle 20, as shown in FIG. 3, the holding device 14 clamps the rod 12. When the lever 34 is pivoted away from the fixed handle 20, as shown in FIG. 4, the holding device 14 releases the rod 12.

The distal end 24 of the fixed handle 20 includes an axially extending opening 40. The opening 40 is defined by a pair of axially extending parallel surfaces 42. The surfaces 42 are interconnected by arcuate upper and lower surfaces 44.

A ratchet member 50 (FIGS. 2A and 3-4) extends into the opening 40. The ratchet mechanism 50 has a first end 52 with an axially extending opening 54. The first end 52 (FIG. 2A) includes parallel side surfaces 58 that are engageable with the surfaces 42 of the fixed handle 20 to guide movement of the ratchet member relative to the fixed handle. The side surfaces 58 are interconnected by an upper arcuate surface 60 and a lower arcuate surface 62. Ratchet teeth 64 extend from the upper arcuate surface 60. Although the ratchet member 50 is shown with six ratchet teeth 64, it is contemplated that the ratchet member may have any number of ratchet teeth.

The distal end 24 of the fixed handle 20 includes an axially extending channel 70 intersecting the opening 40. A pair of circular openings 72 extend through the distal end 24 of the handle 20 and intersect the channel 70. A pin 74 extends through the openings 72 to pivotally connect a locking member 78 to the distal end 24 of the fixed handle 20. The pin 74 extends through the openings 72 in the handle 20 and through an opening 80 in the locking member 78 to pivotally connect the locking member to the handle.

The locking member 78 (FIGS. 2A and 3-4) includes an engaging end 84 that extends from the channel 70 into the opening 40 in the handle 20. The engaging end 84 may engage the teeth 64 on the ratchet member 50. The locking member 78 prevents axial movement of the ratchet member 50 in a proximal direction relative to the handle 20 when the end 84 engages one of the teeth 64.

The locking member 78 has an upwardly extending portion 86 that extends out of the channel 70. A manually engageable portion 88 of the locking member 78 extends in a proximal direction from the upwardly extending portion 84. The manually engageable portion 88 of the locking member 78 is wider than the upwardly extending portion 84. The portion 88 of the locking member 78 may be moved toward the fixed handle 20 to move the end 84 out of engagement with the teeth 64 and permit movement of the ratchet member 50 in the proximal direction relative to the handle.

A spring 92 extends from the manually engageable portion 88 of the pawl 78 into a circular recess 94 in the fixed handle 20. The spring 92 urges the locking member 78 to pivot in a counterclockwise direction relative to the fixed handle 20. Accordingly, the spring 92 urges the end 84 into engagement with the teeth 64 on the ratchet member 50.

The ratchet member 50 (FIGS. 3 and 4) includes a cylindrical linking portion or second end 94 extending from the first end 52 in a proximal direction. The linking portion 94 extends through a cylindrical passage 96 in a mounting portion 98 of the handle 20. The passage 96 extends parallel to the longitudinal axis 21 of the handle 20 from the opening 40 to the recess 26. Accordingly, the linking portion 94 extends through the passage 96 into the recess 26.

The linking portion 94 (FIG. 2A) extends into a channel 102 defined in a first end 104 of a linking member 106. A pin 108 (FIGS. 3 and 4) pivotally connects the linking member 106 to the linking portion 94 of the ratchet member 50. The handle 20 (FIG. 2A) has an opening 109 through which the pin 108 may be inserted to pivotally connect the linking portion 94 to the linking member 106. A second end 110 of the linking member 106 extends into a channel 112 formed in the lever 34. A pivot pin 118 pivotally connects the end 110 of the linking member 106 to the lever 34.

The lever 34 includes an opening 124. The pin 32 extends through the opening 30 in the fixed handle 20 and the opening 124 in the lever 34 to pivotally connect the lever to the fixed handle. When the lever 34 is pivoted away from the handle 20 from a locked position, shown in FIG. 3, toward a release position, shown in FIG. 4, the ratchet member 50 moves in a proximal direction relative to the handle. When the lever 34 is pivoted toward the handle 20 from the release position, shown in FIG. 4, toward the locked position, shown in FIG. 3, the ratchet member 50 moves in a distal direction relative to the handle. Inclined surfaces on the ratchet teeth 64 engage the end 84 of the locking mechanism 78 and cause the locking member to pivot in a clockwise direction against the force applied by the spring 92 as the ratchet member 50 moves in the distal direction.

An axially extending tubular outer sleeve 130 (FIGS. 2A-4) has a first proximal end 132 that extends into the opening 54 in the ratchet member 50. The sleeve 130 has an outer diameter that is slightly smaller than the diameter of the opening 54 in the ratchet member 50. The proximal end 132 of the sleeve 130 is connected to the ratchet member 50 for movement therewith. The proximal end 132 of the sleeve 130 is connected to the ratchet member 150 in any suitable manner, such as by welding. The sleeve 130 defines an axially extending passage 134 extending from the first end 132 to a second distal end 136 of the sleeve. The second end 136 (FIG. 2B) of the sleeve 130 has a cylindrical recess 137 with a diameter larger than the diameter of the passage 134.

A tubular cam member 138 is connected to the second end 136 of the sleeve 130. The cam member 138 has a first proximal end 140 with an outer diameter slightly smaller that the diameter of the recess 137 in the sleeve 130. The first end 140 extends into the recess 137. The proximal end 140 of the cam member 138 is connected to the sleeve 130 in any suitable manner, such as by welding.

The cam member 138 (FIGS. 3 and 4) defines a passage 142 from the first end 140 to a second distal end 144. The passage 142 is a continuation of passage 134 in the sleeve 130. The second end 144 has an outer diameter equal to the outer diameter of the sleeve 130. The second end 144 (FIG. 2B) includes a pair of parallel surfaces 146 defining the passage 142. The surfaces 146 are interconnected by arcuate surfaces 148. A pair of tapered surfaces 150, one of which is shown in FIG. 2B, extend from an end surface 152 of the cam member 138 to the arcuate surfaces 148.

A cylindrical inner tubular member 160 (FIGS. 2A-4) extends through the passage 134 of the sleeve 130 and is fixedly connected to the fixed handle 20. The member 160 has an outer diameter slightly smaller than the diameter of the passage 134. The member 160 (FIGS. 3 and 4) has a first end 162 that extends into a cylindrical opening 164 in the mounting portion 98 of the fixed handle 20. A pair of threaded openings 168 in the mounting portion 98 extend transverse to the opening 164 and intersect the opening 164. A pair of set screws (not shown) threadably engage the openings 168 to clamp the end 162 of the member 160 to the handle 20.

The tubular member 160 defines a passage 172 extending from the first end 162 to a second end 174. The second end 174 has a cylindrical recess 176 with a diameter larger than the diameter of the passage 172. The second end 174 of the member 160 is connected to a pair of clamping members 178 and 180 extending through the passage 142 in the cam member 138. The clamping members 178 and 180 are identical. Accordingly, only one of the clamping members 178 and 180 will be described in detail.

Each of the clamping members 178 and 180 (FIG. 2B) has a first proximal end 182 that extends into the recess 176 in the member 160. The proximal ends 182 are fixedly connected to the member 160. The proximal ends 182 are connected to the member 160 in any suitable manner, such as by welding.

The clamping members 178 and 180 include axially extending channels 184, one of which is shown in FIG. 2B. The channels 184 in the clamping members 178 and 180 define a cylindrical passage 186 (FIGS. 3 and 4) when the clamping members 178 and 180 are connected to the member 160. The passage 186 is a continuation of the passage 172 in the member 160.

The clamping members 178 and 180 (FIG. 2B) include radially outwardly extending clamping portions 188. The clamping portions 188 include outer surfaces 190 that are engageable with the tapered surfaces 150 on the cam member 138. The tapered surfaces 150 on the cam member 138 engage the surfaces 190 to move the clamping members 178 and 180 toward each other.

Each of the clamping members 178 and 180 includes a spherical recess 192. The spherical recesses 192 receive ball portions 196 on jaws 198. Portions of the clamping members 178 and 180 are swaged to retain the ball portions 196 in the spherical recesses 192. The jaws 198 are identical. Accordingly, only one of the jaws 198 will be described in detail. The jaws 198 have clamping portions 202 with part cylindrical surfaces 204 for engaging the rod 12. Each of the jaws 198 has an opening through which a pivot pin 210 extends to pivotally connect the jaws 198 to each other.

When the lever 34 is in the release position, shown in FIG. 4, the lever is pivoted away from the fixed handle 20. The ratchet member 50 is in a release or proximal position. The locking member 78 is disengaged from the teeth 64 on the ratchet member 50. The outer sleeve 130 and cam member 138 are also in a release or proximal position. The tapered surfaces 150 on the cam member 138 are spaced from the surfaces 190 on the clamping members 178 and 180. The jaws 198 may pivot relative to each other. The clamping portions 202 of the jaws 198 may pivot away from each other to receive the rod 12.

After the rod 12 is placed between the clamping portions 202 of the jaws 198, the lever 34 is pivoted toward the fixed handle 20. The ratchet member 50 moves in a distal direction relative to the fixed handle 20, so that one of the teeth 64 engages the locking member 78. The locking member 78 prevents movement of the ratchet member 50 in the proximal direction. The locking member 78 locks the ratchet member in any one of a plurality of positions relative to the fixed handle 20.

The outer sleeve 130 and cam member 138 move with the ratchet member 50 in the distal direction relative to the member 160 and clamping members 178 and 180. The tapered surfaces 150 on the cam member 138 engage the radially outer surfaces 190 on the clamping members 178 and 180. The clamping portions 188 of the clamping members 178 and 180 move toward each other to move the ball portions 196 of the jaws 198 toward each other. Accordingly, the clamping portions 202 of the jaws 198 move toward each other to clamp the rod 12. The locking member 78 engages one of the teeth 64 on the ratchet member 50 to prevent the jaws 198 from pivoting relative to each other to release the rod 12. The amount of clamping force applied by the clamping portions 202 is determined by the position of the lever 34 relative to the handle 20. Accordingly, the clamping force applied by the clamping portions 202 to the rod 12 may be adjusted.

The articulating device 16 includes an articulating member 220 (FIGS. 2B and 3-4) with a radially outwardly extending first distal end 222. The first end 222 is pivotally connected to the pivot pin 210 that pivotally connects the jaws 198 to each other. The first end 222 (FIG. 2B) extends between legs 224 on the pin 210. A pin 226 pivotally connects the end 222 of the articulating member 220 to the legs 224 of the pin 210. It is contemplated that the articulating member 220 may be connected to the pivot pin 210 by a ball and socket joint.

The articulating member 220 (FIGS. 3 and 4) includes a central axially extending portion 228 extending from the first end 222 to a second proximal end 230. The central portion 228 extends axially through the passage 186 defined by the clamping members 178 and 180. The central portion 228 also extends axially through the passage 172 in the member 160.

The second end 230 of the articulating member 220 is fixedly connected to a block member 232 located in the recess 26 in the handle 20. The second end 230 of the articulating member 220 extends through an opening 234 in the block member 232. A threaded opening 235 extends transverse to the opening 234 and intersects the opening 234. A set screw (not shown) threadably engages the opening 235 to clamp the articulating member 220 to the block member 232.

The block member 232 (FIG. 2A) has a pair of threaded openings 240 extending through an upper surface 242 of the block member. The threaded openings 240 extend on opposite sides of the opening 234 so that the threaded openings 240 do not intersect the opening 234. A slider 248 is connected to the block member 232. The slider 248 has openings 250 (FIGS. 1 and 2A) through which fasteners (not shown) extend to connect the slider 248 to the block member 232.

The fasteners (not shown) also extend through a slot 260 (FIG. 2A) in the fixed handle 20 to connect the slider 248 to the block member 232. The slot 260 extends through a planar upper surface 262 of the handle 20. The surface 262 partially defines a rectangular recess 264 in the fixed handle 20. The recess 264 is defined by axially extending parallel surfaces 266. A proximal surface 268 extends between the parallel surfaces 266. A distal surface 270 (FIGS. 3 and 4) extends generally parallel to the surface 268 and interconnects the parallel surfaces 266. The parallel surfaces 266 engage parallel side surfaces 280, one of which is shown in FIG. 2A, on the slider 248 to guide movement of the slider relative to the fixed handle 20.

The articulating device 16 may be actuated to rotate the rod 12 from a first insertion position, shown in FIG. 3, extending parallel to the axis 21 of the handle 20 to a second articulated position, shown in FIG. 4, extending transverse to the axis of the handle and the first position. When the slider 248 is in a proximal or insertion position, as shown in FIG. 3, the articulating member 220 is also in an insertion or proximal position. The rod 12 extends parallel to the longitudinal axis 21 of the handle 20.

When the slider 248 is moved in a distal direction relative to the fixed handle 20 from the position shown in FIG. 3 to the position shown in FIG. 4, the articulating member 220 moves axially in a distal direction relative to the handle 20 and the clamping members 178 and 180. The movement of the articulating member 220 causes the jaws 198 to pivot relative to the clamping members 178 and 180 about an axis of the ball portions 196. Accordingly, the rod 12 rotates about the axis of the ball portions 196.

The surgical tool 10 may be used to move the rod 12 into the body of a patient during a minimally invasive surgical procedure. The rod 12 may be moved through a passage in an access port, tubular structure, retractor, or cannula 300 into the body of the patient. One suitable cannula 300 is similar to a cannula shown in U.S. Pat. No. 6,187,000, incorporated herein entirely by reference. U.S. patent application Ser. No. 09/772,605, filed Jan. 30, 2001, incorporated herein entirely by reference, discloses other cannula structures that may be used. U.S. patent application Ser. No. 10/926,840, filed Aug. 26, 2004, is incorporated herein entirely by reference. The cannula 300 may be a tubular structure defining a passage having an expandable distal end. Surgical instruments and an endoscope may be inserted into a patient's body through the passage during a minimally invasive surgery. It is contemplated that the access port, tubular structure, retractor, or cannula 300 may have any suitable configuration.

The surgical tool 10 may be used to place the rod 12 into connectors 314 connected to vertebrae V of a spinal column during a minimally invasive surgery. The connectors 314 may be of any desired configuration. One suitable connector 314 is similar to a connector shown in U.S. patent application Ser. No. 10/075,668, filed Feb. 13, 2002, incorporated herein entirely by reference. Other suitable connectors are shown in PCT Application No. US03/04361, filed Jan. 13, 2004, incorporated herein entirely by reference.

The connectors 314 include housings 316 pivotable relative to fasteners 318. The housings 316 have passages (not shown) through which the rod 12 extends when inserted into the housings. Set screws (not shown) threadably engage the housings 316 to clamp the rod 12 to the housings and prevent relative movement between the housings and the fasteners 318.

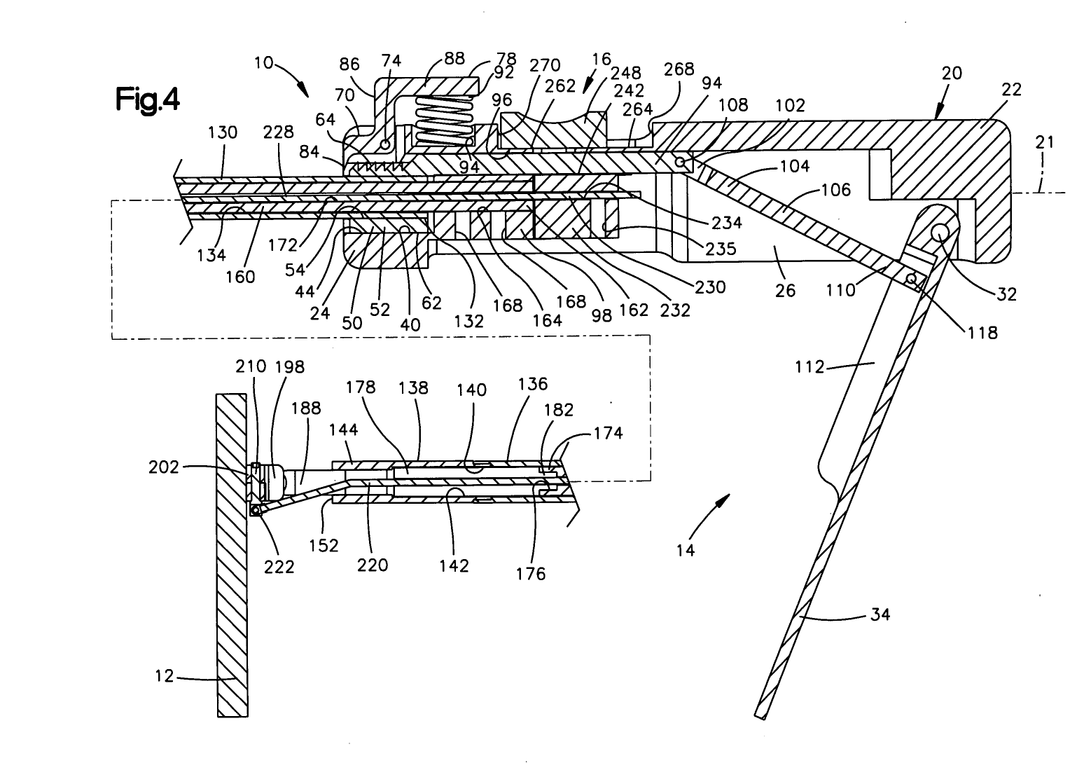

During the minimally invasive surgery, the rod 12 is held by the surgical tool 10 in the first or insertion position, as shown in FIGS. 3 and 5. The lever 34 is pivoted toward the handle 20 to cause the jaws 198 to clamp the rod 12. The locking member 78 prevents the jaws 198 from releasing the rod 12. The rod 12 is moved through the passage in the cannula 300 in the first position with the surgical tool 10, as shown in FIG. 5.

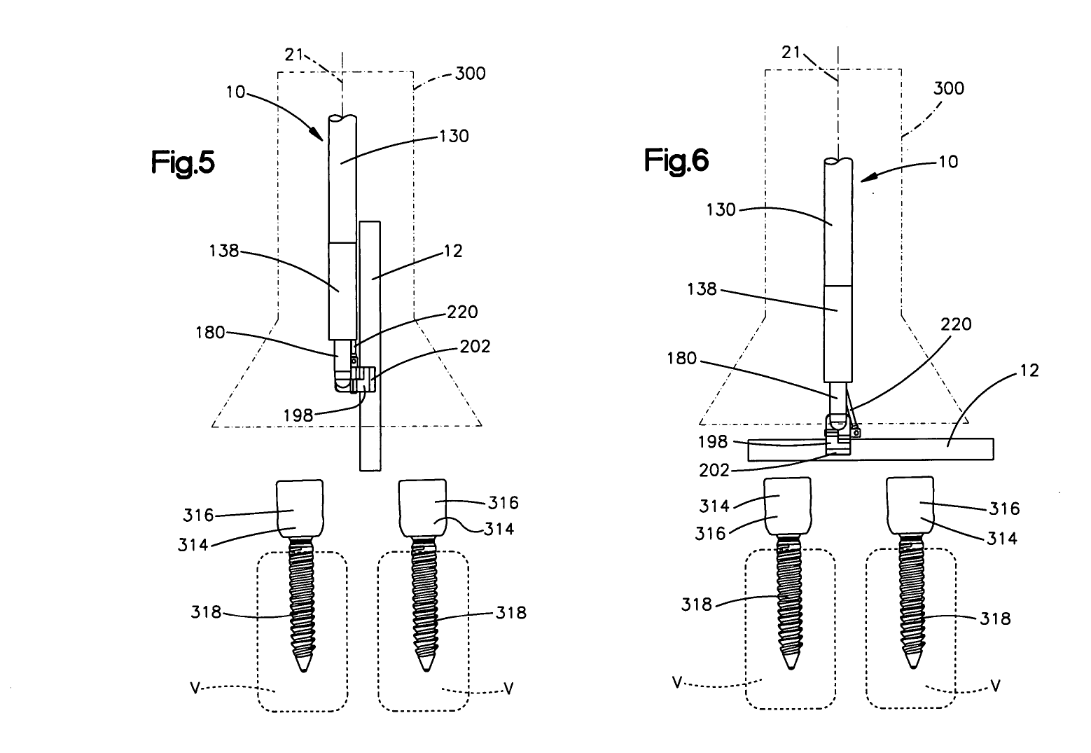

Once the rod 12 has been moved through the cannula 300 to adjacent the distal end of the cannula, the slider 248 is moved in a distal direction relative to the handle 20. The rod 12 rotates from the first position into the second position, shown in FIGS. 4 and 6, extending transverse to the first position without releasing the rod 12. After the rod 12 is rotated to the second position, the rod is inserted into the connectors 314. The locking member 78 is pressed and the lever 34 is pivoted away from the handle 20 to the position shown in FIG. 4 to release the rod 12. The set screws (not shown) are then threaded into the connectors 314 to clamp the rod 12 to the housings 316 and retain the vertebrae V in a desired spatial relationship.

Although the surgical tool 10 is described as inserting a rod 12 into the body of the patient, it is contemplated that the surgical tool may insert any suitable longitudinal member, such as a plate or a hexagonal-shaped rod.

It will be understood that the above description of the present invention is susceptible to various modifications, changes, and adaptations, and the same are intended to be comprehended within the meaning and range of equivalents, of the appended claims. The presently disclosed embodiments, are considered in all respects to be illustrative and not restrictive. The scope of the invention indicated by the appended claims, rather than the foregoing description, and all changes that come within the meaning and range of equivalence thereof are intended to be embraced therein.

1. A method for inserting a longitudinal member into a body of a patient, said method comprising:
holding the longitudinal member in a first position with a surgical tool;
moving the longitudinal member through an access port in the first position with the surgical tool;
rotating the longitudinal member from the first position into a second position extending transverse to the first position without releasing the longitudinal member from the surgical tool.

2. A method as defined in claim 1 including axially moving a member of the surgical tool to clamp the longitudinal member.

3. A method as defined in claim 2 including moving a lever of the surgical tool relative to a handle of the surgical tool to axially move the member.

4. A method as defined in claim 3 including pivoting the lever toward the handle to axially move the member.

5. A method as defined in claim 2 including axially moving the member in a distal direction to clamp the longitudinal member.

6. A method as defined in claim 1 including axially moving an articulating member of the surgical tool to rotate the longitudinal member from the first position into the second position.

7. A method as defined in claim 6 including axially moving the articulating member in a distal direction to rotate the longitudinal member from the first position into the second position.

8. A method as defined in claim 6 including moving a slider of the surgical tool relative to a handle of the surgical tool in a direction parallel to a longitudinal axis of the surgical tool to rotate the longitudinal member from the first position into the second position.

9. A method as defined in claim 1 including moving a locking member of the surgical tool relative to a handle of the surgical tool to release the longitudinal member from the surgical tool.

10. A method as defined in claim 9 including moving a lever of the surgical tool relative to the handle to release the longitudinal member from the surgical tool.

11. A method as defined in claim 9 including pivoting the locking member relative to the handle to release the longitudinal member from the surgical tool.

12. A method as defined in claim 1 including adjusting the clamping force applied by clamping portions of the surgical tool to the longitudinal member.

13. A surgical tool for inserting a longitudinal member into a body of a patient, said surgical tool including:
a holding device for holding the longitudinal member in a first position;
an articulating device for rotating the longitudinal member from the first position into a second position extending transverse to the first position without releasing the longitudinal member from the surgical tool.

14. A surgical tool as defined in claim 13 wherein said surgical tool includes an axially movable member configured to cause said surgical tool to clamp the longitudinal member.

15. A surgical tool as defined in claim 14 wherein said surgical tool includes a lever movable relative to a handle to axially move said member.

16. A surgical tool as defined in claim 15 wherein said lever is pivotable toward said handle to axially move said member.

17. A surgical tool as defined in claim 16 wherein said lever axially moves said member in a distal direction to cause said surgical too to clamp the longitudinal member.

18. A surgical tool as defined in claim 13 wherein said surgical tool includes an axially movable articulating member configured to rotate the longitudinal member from the first position into the second position.

19. A surgical tool as defined in claim 18 wherein said articulating member is movable in a distal direction to rotate the longitudinal member from the first position into the second position.

20. A surgical tool as defined in claim 18 wherein said surgical tool includes a slider movable relative to a handle in a direction extending parallel to a longitudinal axis of said surgical tool to rotate the longitudinal member from the first position into the second position.

21. A surgical tool as defined in claim 13 wherein said surgical tool includes a locking member movable relative to a handle to release the longitudinal member from said surgical tool.

22. A surgical tool as defined in claim 21 wherein said surgical tool includes a lever movable relative to said handle to release the longitudinal member from the surgical tool.

23. A surgical tool as defined in claim 21 wherein said locking member is pivotable relative to said handle to release the longitudinal member from the surgical tool.

24. A surgical tool as defined in claim 13 wherein said surgical tool includes a member configured to adjust a clamping force applied by clamping portions of said surgical tool to the longitudinal member.
